(19) United States
(12) Patent Application Publication
(10) Pub. No.: US 2024/0307180 A1
(43) Pub. Date: Sep. 19, 2024

(54) STENT DELIVERY DEVICE WITH SECONDARY VISUAL INDICATOR TO ENSURE PROPER POSITIONING TO SEE PRIMARY VISUAL INDICATOR

(71) Applicant: BOSTON SCIENTIFIC SCIMED, INC., MAPLE GROVE, MN (US)

(72) Inventor: Chris Cullen, Galway (IE)

(73) Assignee: BOSTON SCIENTIFIC SCIMED, INC., MAPLE GROVE, MN (US)

(21) Appl. No.: 18/599,811

(22) Filed: Mar. 8, 2024

Related U.S. Application Data
(60) Provisional application No. 63/452,540, filed on Mar. 16, 2023.

(51) Int. Cl. A61F 2/24; A61M 25/01

(52) U.S. Cl. CPC A61F 2/2436; A61F 2/2418; A61F 2250/0098; A61M 25/0108

(57) ABSTRACT

An implant delivery system for delivering a replacement heart valve implant to a native valve annulus may include an elongate shaft assembly including an implant holding portion configured to constrain a replacement heart valve implant in a radially collapsed configuration. The elongate shaft assembly includes a primary visual indicator configured to be visible under fluoroscopy with an imaging device. The elongate shaft assembly includes a secondary visual indicator configured to be visible under fluoroscopy with the imaging device when the imaging device is aligned with a reference plane associated with the native valve annulus. The secondary visual indicator is not visible under fluoroscopy with the imaging device when the imaging device is misaligned with the reference plane associated with the native valve annulus.

### CROSS-REFERENCE TO RELATED APPLICATIONS

This application claims the benefit of priority of U.S. Provisional Application No. 63/452,540 filed Mar. 16, 2023, the entire disclosure of which is hereby incorporated by reference.

### TECHNICAL FIELD

The disclosure relates generally to medical devices and more particularly to medical devices that are adapted for implanting stents and medical devices including a stent component.

### BACKGROUND

A wide variety of intracorporeal medical devices have been developed for medical use including, artificial heart valves for repair or replacement of diseased heart valves. The artificial heart valves need to be precisely aligned relative to a native valve annulus when implanted. Of the known medical devices and methods, each has certain advantages and disadvantages. There is an ongoing need to provide alternative medical devices as well as alternative methods for manufacturing and using medical devices.

### SUMMARY

In one example, an implant delivery system for delivering a replacement heart valve implant to a native valve annulus may comprise an elongate shaft assembly including an implant holding portion comprising a proximal sheath and a distal sheath. The implant holding portion is configured to constrain a replacement heart valve implant in a radially collapsed configuration. The elongate shaft assembly includes a primary visual indicator configured to be visible under fluoroscopy with an imaging device. The elongate shaft assembly includes a secondary visual indicator configured to be visible under fluoroscopy with the imaging device when the imaging device is aligned with a reference plane associated with the native valve annulus. The secondary visual indicator is not visible under fluoroscopy with the imaging device when the imaging device is misaligned with the reference plane associated with the native valve annulus.

In addition or alternatively to any example disclosed herein, a desired insertion depth of the replacement heart valve implant relative to the reference plane associated with the native valve annulus is about 7 millimeters from the reference plane associated with the native valve annulus to an inflow end of the replacement heart valve implant.

In addition or alternatively to any example disclosed herein, the primary visual indicator is configured to define an actual insertion depth of the replacement heart valve implant relative to the reference plane associated with the native valve annulus.

In addition or alternatively to any example disclosed herein, when the secondary visual indicator is visible under fluoroscopy and the primary visual indicator is aligned with the reference plane under fluoroscopy, the actual insertion depth is within 10% of the desired insertion depth.

In addition or alternatively to any example disclosed herein, when the secondary visual indicator is not visible under fluoroscopy and the primary visual indicator is aligned with the reference plane under fluoroscopy, the actual insertion depth deviates from desired insertion depth by at least 10%.

In addition or alternatively to any example disclosed herein, the elongate shaft assembly includes a stent holder configured to engage an expandable framework of the replacement heart valve implant in the radially collapsed configuration.

In addition or alternatively to any example disclosed herein, the secondary visual indicator comprises an annular recess formed within the stent holder.

In addition or alternatively to any example disclosed herein, when the imaging device is aligned with the reference plane associated with the native valve annulus and the primary visual indicator is aligned with the reference plane under fluoroscopy, the annular recess is visible to the imaging device as a void oriented generally parallel to the reference plane.

In addition or alternatively to any example disclosed herein, when the imaging device is misaligned with the reference plane associated with the native valve annulus and the primary visual indicator is aligned with the reference plane under fluoroscopy, the annular recess is at least partially obscured from viewing by the imaging device.

In addition or alternatively to any example disclosed herein, the annular recess has a width of 0.3 millimeters to 0.4 millimeters.

In addition or alternatively to any example disclosed herein, the annular recess has a radial depth defining a remaining radial material thickness of 0.07+/−0.01 millimeters.

In addition or alternatively to any example disclosed herein, the primary visual indicator is axially spaced apart from the secondary visual indicator.

In addition or alternatively to any example disclosed herein, a replacement heart valve system may comprise a replacement heart valve implant comprising an expandable framework and a plurality of valve leaflets secured to the expandable framework, the expandable framework being configured to shift between a radially collapsed configuration and a radially expanded configuration; and an implant delivery system for delivering the replacement heart valve implant to a native valve annulus. The implant delivery system may comprise an elongate shaft assembly including an implant holding portion comprising a proximal sheath and a distal sheath. The implant holding portion is configured to constrain the replacement heart valve implant in the radially collapsed configuration. The elongate shaft assembly includes a primary visual indicator configured to be visible under fluoroscopy with an imaging device. The elongate shaft assembly includes a secondary visual indicator configured to be visible under fluoroscopy with the imaging device when the imaging device is aligned with a reference plane associated with the native valve annulus. The secondary visual indicator is not visible under fluoroscopy with the imaging device when the imaging device is misaligned with the reference plane associated with the native valve annulus.

In addition or alternatively to any example disclosed herein, the implant delivery system is configured to cooperate with the imaging device to position the replacement heart valve implant within the native valve annulus.

In addition or alternatively to any example disclosed herein, the implant delivery system is configured to position the replacement heart valve implant within the native valve annulus at an actual insertion depth relative to the reference plane associated with native valve annulus that is within 10% of a desired insertion depth relative to the reference plane associated with native valve annulus when the primary visual indicator and the secondary visual indicator are both visible to the imaging device under fluoroscopy.

In addition or alternatively to any example disclosed herein, when the primary visual indicator is aligned with the reference plane, the secondary visual indicator is only visible to the imaging device under fluoroscopy when the imaging device is aligned with the reference plane associated with the native valve annulus.

In addition or alternatively to any example disclosed herein, method of delivering a replacement heart valve implant to a native valve annulus may comprise: advancing an implant delivery system to a position adjacent the native valve annulus, wherein the replacement heart valve implant is constrained within an implant holding portion of the implant delivery system; positioning an imaging device in alignment with a reference plane associated with the native valve annulus; aligning a primary visual indicator of the implant delivery system with the reference plane under fluoroscopy using the imaging device; visualizing a secondary visual indicator of the implant delivery system under fluoroscopy using the imaging device when the primary visual indicator is aligned with the reference plane to verify alignment of the imaging device with the reference plane; and deploying the replacement heart valve implant from the implant delivery system within the native valve annulus.

In addition or alternatively to any example disclosed herein, the secondary visual indicator is configured to be visible under fluoroscopy with the imaging device when the imaging device is aligned with the reference plane associated with the native valve annulus and the primary visual indicator is aligned with the reference plane, and the secondary visual indicator is not visible under fluoroscopy with the imaging device when the imaging device is misaligned with the reference plane associated with the native valve annulus and the primary visual indicator is aligned with the reference plane.

In addition or alternatively to any example disclosed herein, when the primary visual indicator is aligned with the reference plane, the secondary visual indicator is only visible under fluoroscopy with the imaging device when the imaging device is not oriented at an oblique angle intersecting the reference plane associated with the native valve annulus.

In addition or alternatively to any example disclosed herein, the secondary visual indicator is an annular recess formed in a stent holder of the implant delivery system, the stent holder being configured to engage an expandable framework of the replacement heart valve implant when the replacement heart valve implant is constrained within the implant holding portion.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cutaway view illustrating selected aspects of a replacement heart valve implant positioned within a native valve annulus of a heart;

FIG. 2 illustrates selected aspects of a delivery system for delivering a replacement heart valve implant;

FIG. 3 is a partial cutaway view illustrating selected aspects related to using an imaging device when delivering a replacement heart valve implant to the heart; and

FIG. 4 is a partial cutaway view illustrating examples of incorrect alignment of the imaging device of FIG. 3;

FIGS. 5, 5A, and 5B schematically illustrate the parallax effect;

FIG. 6 is a partial cross-sectional view illustrating selected aspects of a portion of a delivery system according to the disclosure;

FIGS. 7A-7B illustrate selected aspects of a stent holder of the delivery system according to the disclosure;

FIG. 8A is a fluoroscopic image of an assembly with a secondary visual indicator visible to the imaging device;

FIG. 8B is a fluoroscopic image of the assembly of FIG. 8A with the secondary visual indicator obscured to the imaging device; and

FIG. 9 is a fluoroscopic image illustrating a primary visual indicator and the secondary visual indicator of the disclosure relative to a native valve annulus when the imaging device is properly aligned with the native valve annulus.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

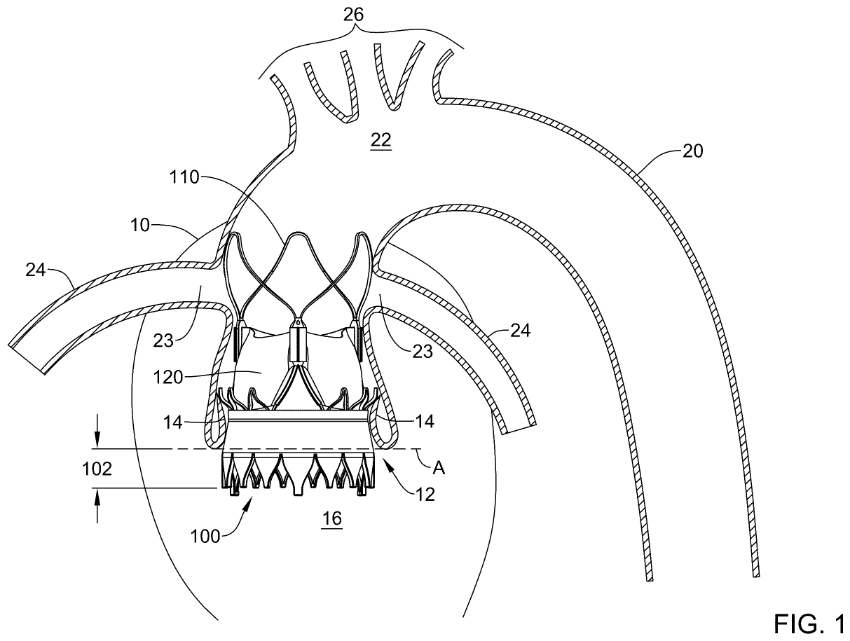

FIG. 1 illustrates a schematic partial cut-away view of a portion of a patient's heart 10 including the aortic valve 12 having native valve leaflets 14 disposed within and/or extending from the native valve annulus, a left ventricle 16, and certain connected vasculature, such as the aorta 20 connected to the aortic valve 12 of the patient's heart 10 by the aortic arch 22, the coronary arteries 24, the ostia 23 of the coronary arteries 24, and other large arteries 26 (e.g., subclavian arteries, carotid arteries, brachiocephalic artery) that extend from the aortic arch 22 to important internal organs. FIG. 1 shows plane A aligned with the native valve annulus of the aortic valve 12. For the purpose of this disclosure, the discussion herein is directed toward use in treating the aortic valve 12 and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure.

FIG. 1 further illustrates selected aspects of a replacement heart valve implant 100 positioned within the aortic valve 12 and/or the native valve annulus of the aortic valve 12, wherein the native valve annulus is identified and/or approximated by a reference plane A associated with the native valve annulus. It should be appreciated that the replacement heart valve implant 100 can be any type of replacement heart valve (e.g., a mitral valve, an aortic valve, etc.). In use, the replacement heart valve implant 100 may be implanted (e.g., surgically or through transcatheter delivery) in a mammalian heart. The replacement heart valve implant 100 can be configured to allow one-way flow through the replacement heart valve implant 100 from an inflow end to an outflow end. In some embodiments, a desired insertion depth 102 of the replacement heart valve implant 100 relative to the reference plane A associated with the native valve annulus may be about 7 millimeters from the reference plane A associated with the native valve annulus to the inflow end of the replacement heart valve implant 100. This is only an example and other configurations and/or desired insertion depths are also contemplated. In some embodiments, different sizes of the replacement heart valve implant 100 may have different desired insertion depths.

The replacement heart valve implant 100 may include an expandable framework 110 defining a central lumen. In some embodiments, the expandable framework 110 may have a substantially circular cross-section. In some embodiments, the expandable framework 110 can have a non-circular (e.g., D-shaped, elliptical, etc.) cross-section. Some suitable but non-limiting examples of materials that may be used to form the expandable framework 110, including but not limited to metals and metal alloys, composites, ceramics, polymers, and the like, are described below. The replacement heart valve implant 100 and/or the expandable framework 110 may be configured to shift between a radially collapsed configuration and a radially expanded configuration. In some embodiments, the expandable framework 110 may be self-expanding. In some embodiments, the expandable framework 110 may be self-biased toward the radially expanded configuration. In some embodiments, the expandable framework 110 may be mechanically expandable. In some embodiments, the expandable framework 110 may be balloon expandable. Other configurations are also contemplated.

In some embodiments, the expandable framework 110 may define a lower crown proximate and/or at the inflow end, an upper crown proximate and/or at the outflow end, and a plurality of stabilization arches extending downstream from the outflow end. In some embodiments, the plurality of stabilization arches may extend downstream of and/or away from the upper crown in a direction opposite the lower crown. In some embodiments, the upper crown may be disposed longitudinally and/or axially between the lower crown and the plurality of stabilization arches. The expandable framework 110 may define a central lumen extending therethrough.

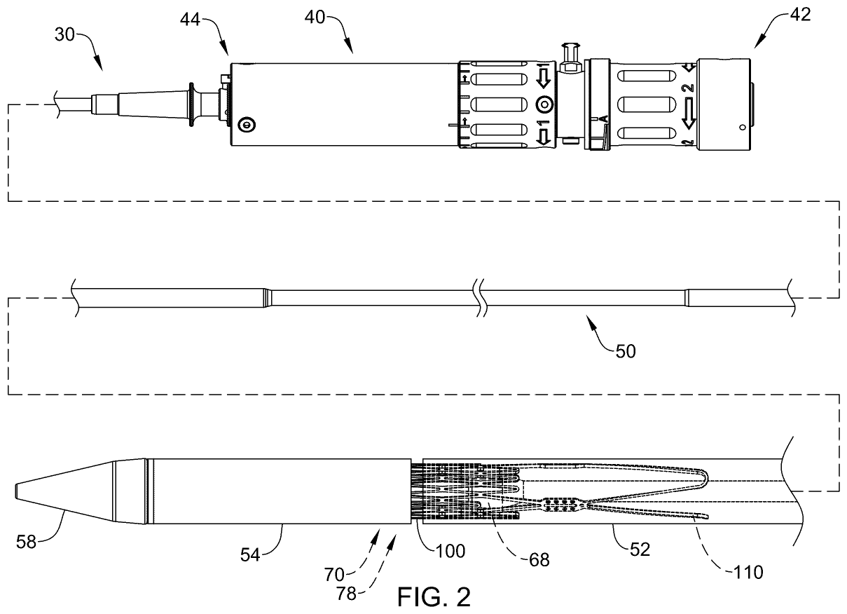

In some embodiments, the replacement heart valve implant 100 may include a proximal portion and a distal portion. In some embodiments, orientation of the replacement heart valve implant 100 may be related to an implant delivery system 30 (e.g., FIG. 2) and/or a direction of implantation relative to a target site. In some embodiments, the proximal portion may include the outflow end and/or the plurality of stabilization arches. In some embodiments, the proximal portion may include the upper crown. In some embodiments, the distal portion may include the inflow end and/or the lower crown. Other configurations are also contemplated.

In some embodiments, the replacement heart valve implant 100 may include a plurality of valve leaflets 120 disposed within the central lumen. The plurality of valve leaflets 120 may be coupled, secured, and/or fixedly attached to the expandable framework 110 at a plurality of commissures. The plurality of valve leaflets 120 may be configured to shift between an open position and a closed position. The plurality of valve leaflets 120 may be configured to substantially restrict fluid flow through the replacement heart valve implant 100 in the closed position. The plurality of valve leaflets 120 may move apart from each other in the open position to permit fluid flow through the replacement heart valve implant 100.

In some embodiments, the plurality of valve leaflets 120 may be comprised of a polymer, such as a thermoplastic polymer. In some embodiments, the plurality of valve leaflets 120 may include at least 50 percent by weight of a polymer. In some embodiments, the plurality of valve leaflets 120 may be formed from bovine pericardial or other living tissue. Other configurations and/or materials are also contemplated.

In some embodiments, the replacement heart valve implant 100 may include an inner skirt disposed on and/or extending along an inner surface of the expandable framework 110. In at least some embodiments, the inner skirt may be fixedly attached to the expandable framework 110. The inner skirt may direct fluid, such as blood, flowing through the replacement heart valve implant 100 toward the plurality of valve leaflets 120. In at least some embodiments, the inner skirt may be fixedly attached to and/or integrally formed with the plurality of valve leaflets 120. The inner skirt may ensure the fluid flows through the central lumen of the replacement heart valve implant 100 and does not flow around the plurality of valve leaflets 120 when they are in the closed position.

In some embodiments, the replacement heart valve implant 100 may include an outer skirt disposed on and/or extending along an outer surface of the expandable framework 110. In some embodiments, the outer skirt may be disposed at and/or adjacent the lower crown. The outer skirt may ensure the fluid flows through the replacement heart valve implant 100 and does not flow around the replacement heart valve implant 100 (e.g., between the expandable framework 110 and the vessel wall).

In some embodiments, the inner skirt and/or the outer skirt may include a polymer, and/or may include at least 50 percent by weight of a polymer. In some embodiments, the inner skirt and/or the outer skirt may be substantially impervious to fluid. In some embodiments, the inner skirt and/or the outer skirt may be formed from a thin tissue (e.g., bovine pericardial, etc.), a coated fabric material, or a nonporous and/or impermeable fabric material. Other configurations are also contemplated. Some suitable but non-limiting examples of materials that may be used to form the inner skirt and/or the outer skirt including but not limited to polymers, composites, and the like, are described below.

In some embodiments, the inner skirt and/or the outer skirt may seal one of, some of, a plurality of, or each of a plurality of interstices formed in the expandable framework 110. In at least some embodiments, sealing the interstices may be considered to prevent fluid from flowing through the interstices of the expandable framework 110. In some embodiments, the inner skirt and/or the outer skirt may be attached to the expandable framework 110 using one or more methods including but not limited to tying with sutures or filaments, adhesive bonding, melt bonding, embedding or over molding, welding, etc.

In some embodiments, the expandable framework 110 and/or the replacement heart valve implant 100 may have an outer extent of about 23 millimeters (mm), about 25 mm, about 27 mm, about 30 mm, etc. in an unconstrained configuration (e.g., in the radially expanded configuration). In some embodiments, the expandable framework 110 and/or the replacement heart valve implant 100 may have an outer extent of about 10 mm, about 9 mm, about 8 mm, about 7 mm, about 6 mm, etc. in the radially collapsed configuration. Other configurations are also contemplated.

FIG. 2 illustrates selected aspects of a replacement heart valve system including the replacement heart valve implant 100 and an implant delivery system 30 compatible with and/or usable with the replacement heart valve implant 100. It should be noted that FIG. 2 includes at least one change of scale (e.g., all parts of the figure are not drawn to the same scale) to improve viewability and show additional detail of selected aspects of the implant delivery system 30. Additionally, the expandable framework 110 is shown in the radially collapsed configuration but some elements of the replacement heart valve implant 100 are not shown to improve clarity.

The implant delivery system 30 may include a handle 40 and an elongate shaft assembly 50 extending distally from the handle 40. The handle 40 may include a first end 42 and a second end 44 opposite the first end 42. The elongate shaft assembly 50 may extend distally from the second end 44 of the handle 40. The handle 40 may include one or more rotatable knobs. In some embodiments, the one or more rotatable knobs may include a first rotatable knob and a second rotatable knob. In at least some embodiments, the first rotatable knob and/or the second rotatable knob may be configured to rotate about a central longitudinal axis of the implant delivery system 30 and/or the handle 40.

In some embodiments, a distal portion of the implant delivery system 30 and/or the elongate shaft assembly 50 may include an implant holding portion configured to engage with and/or constrain the replacement heart valve implant 100 and/or the expandable framework 110 in the radially collapsed configuration. The elongate shaft assembly 50 may include an outer tubular member extending distally from the handle 40 and an inner shaft extending distally from the handle 40 within the outer tubular member to a distal tip 58 disposed distal of the implant holding portion. In some embodiments, the implant holding portion may include a proximal sheath 52 and a distal sheath 54. In some embodiments, the inner shaft may be slidably disposed within a lumen of the outer tubular member. In some embodiments, the inner shaft may be fixedly attached to the distal sheath 54 and/or the distal tip 58. In some embodiments, the distal sheath 54 may be fixedly attached to the distal tip 58. In some embodiments, the distal sheath 54 may extend proximally from the distal tip 58. In some embodiments, the inner shaft may include and/or at least partially define a guidewire lumen extending therethrough. In some embodiments, the guidewire lumen may extend through the handle 40.

In some embodiments, the handle 40 may be configured to manipulate and/or translate the proximal sheath 52 and/or the distal sheath 54 relative to each other. In some embodiments, the handle 40 may be configured to manipulate and/or translate the inner shaft relative to the elongate shaft assembly 50, the outer tubular member, and/or the proximal sheath 52.

During delivery of the replacement heart valve implant 100 to a treatment site, the replacement heart valve implant 100 may be disposed at least partially within the proximal sheath 52 and/or the distal sheath 54 in the radially collapsed configuration in a closed configuration of the implant holding portion. In some embodiments, the proximal sheath 52 and/or the distal sheath 54 may collectively define the implant holding portion of the implant delivery system 30. In some embodiments, the implant holding portion may be configured to constrain the replacement heart valve implant 100 in the radially collapsed configuration when the implant holding portion is in the closed configuration. In some embodiments, the replacement heart valve implant 100 may be releasably coupled to the inner shaft and/or a stent holder 70.

In some embodiments, the proximal sheath 52 may be configured to cover the proximal portion of the replacement heart valve implant 100 in the radially collapsed configuration when the implant holding portion is in the closed configuration, and the distal sheath 54 may be configured to cover the distal portion of the replacement heart valve implant 100 in the radially collapsed configuration when the implant holding portion is in the closed configuration. In some embodiments, the replacement heart valve implant 100 and/or the expandable framework 110 may be constrained in the radially collapsed configuration by the proximal sheath 52 and the distal sheath 54 in the closed configuration of the implant holding portion. In some embodiments, the proximal sheath 52 may be disposed adjacent to the distal sheath 54 in the closed configuration. In some embodiments, the proximal sheath 52 may abut the distal sheath 54 in the closed configuration. In some embodiments, the proximal sheath 52 may be axially spaced apart from the distal sheath 54 in the closed configuration. In some embodiments, the proximal sheath 52 may be axially spaced apart from the distal sheath 54 in the closed configuration by less than 20% of an overall length of the replacement heart valve implant 100 and/or the expandable framework 110. In some embodiments, the proximal sheath 52 may be axially spaced apart from the distal sheath 54 in the closed configuration by less than 15% of an overall length of the replacement heart valve implant 100 and/or the expandable framework 110. In some embodiments, the proximal sheath 52 may be axially spaced apart from the distal sheath 54 in the closed configuration by less than 10% of an overall length of the replacement heart valve implant 100 and/or the expandable framework 110. In some embodiments, the proximal sheath 52 may be axially spaced apart from the distal sheath 54 in the closed configuration by less than 5% of an overall length of the replacement heart valve implant 100 and/or the expandable framework 110. Other configurations are also contemplated.

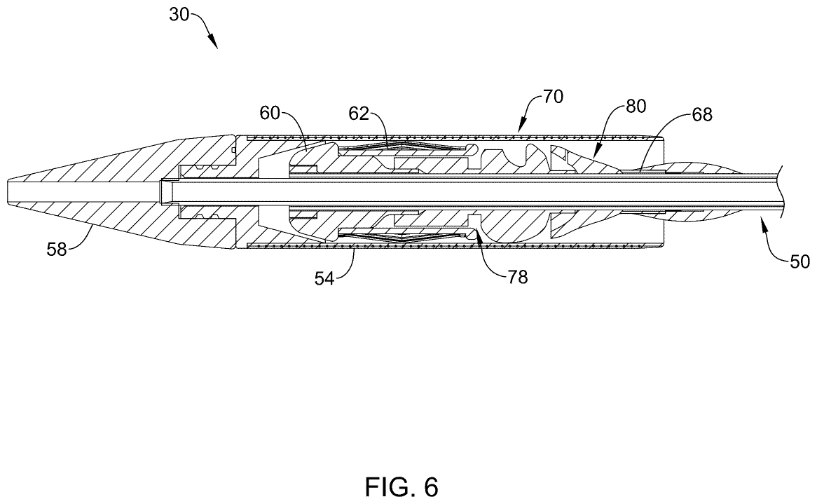

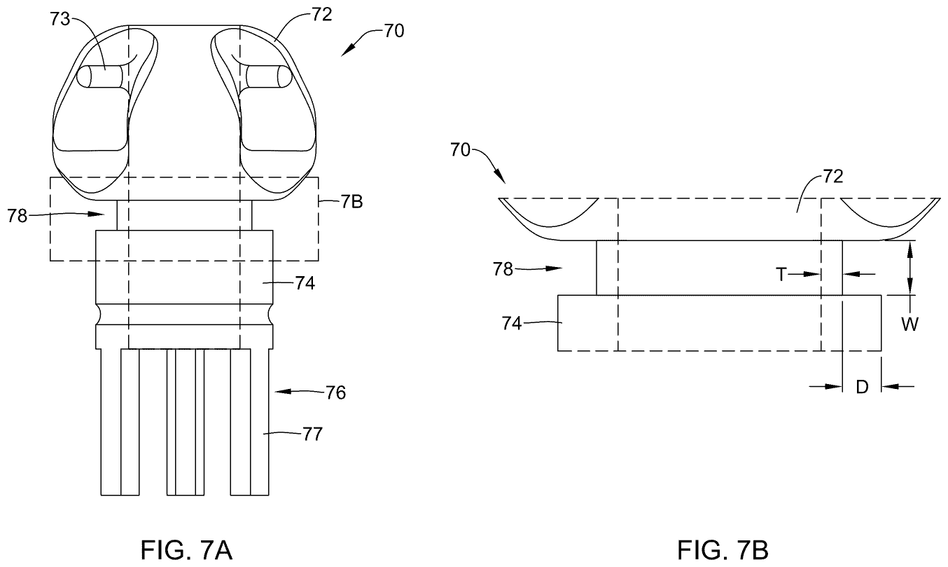

In some embodiments, the implant holding portion and/or the elongate shaft assembly 50 may include the stent holder 70, which is shown in more detail in FIGS. 6-7B. In at least some embodiments, the stent holder 70 may be fixedly attached to the elongate shaft assembly 50. In some embodiments, the stent holder 70 may be integrally formed with the elongate shaft assembly 50. In some embodiments, the stent holder 70 may be configured to engage the expandable framework 110 in the radially collapsed configuration. In some embodiments, the stent holder 70 may include at least one projection 73 (e.g., FIG. 7A) configured to engage the expandable framework 110 in the radially collapsed configuration.

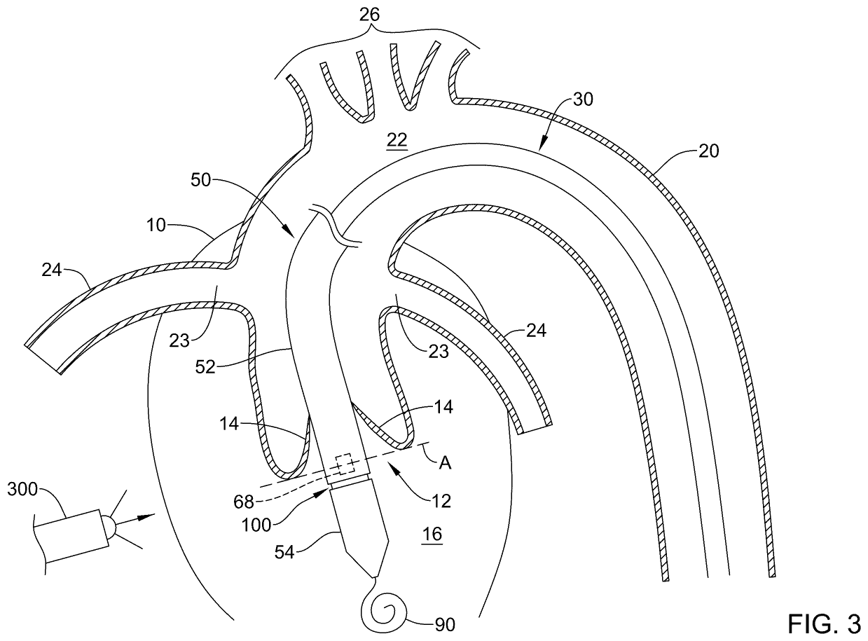

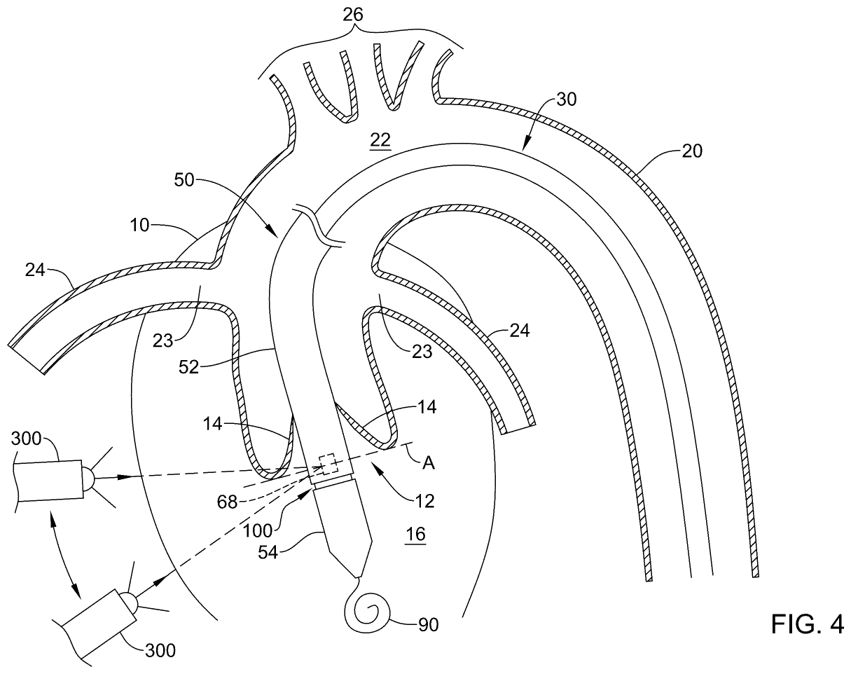

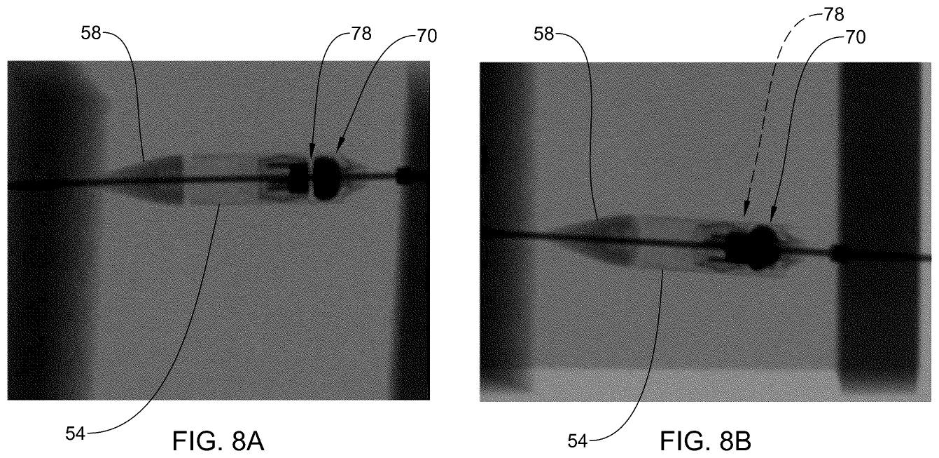

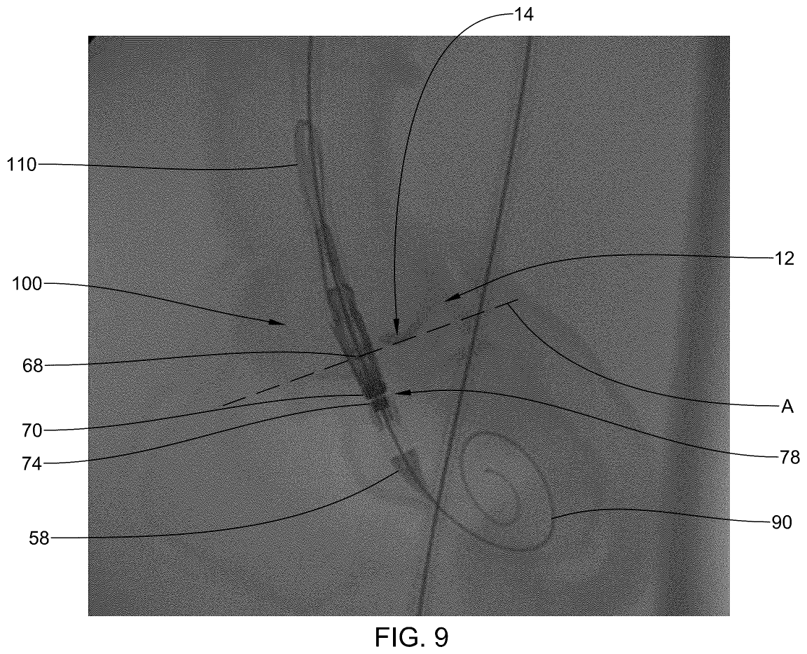

Returning to FIG. 2, the implant delivery system 30 and/or the elongate shaft assembly 50 may include a primary visual indicator 68 configured and/or adapted to be visible under fluoroscopy with an imaging device 300 (e.g., FIGS. 3-4). Other imaging means suitable for use with transcatheter surgical procedures are also contemplated. In some embodiments, the implant delivery system 30 and/or the elongate shaft assembly 50 may include a secondary visual indicator 78 (e.g., FIGS. 6-7B) configured and/or adapted to be visible under fluoroscopy with the imaging device 300 (e.g., FIGS. 3-4), as shown in the fluoroscopic images of FIGS. 8-9. In some embodiments, the stent holder 70 may include the secondary visual indicator 78. In at least some embodiments, the primary visual indicator 68 may be axially spaced apart from the secondary visual indicator 78. The implant delivery system 30 may be configured to cooperate with the imaging device 300 to position the replacement heart valve implant 100 within the native valve annulus.

FIG. 3 illustrates selected aspects related to delivering the replacement heart valve implant 100 to the native valve annulus. In use, the implant delivery system 30 may be advanced percutaneously through the vasculature to a position adjacent to the treatment site (e.g., the native valve annulus). For example, the implant delivery system 30 may be advanced through the vasculature and across the aortic arch 22 to a position adjacent to the aortic valve 12. Alternative approaches to treat a defective aortic valve and/or other heart valve(s) are also contemplated with the implant delivery system 30.

The desired insertion depth 102 (e.g., FIG. 1) is selected to maximize radially outward force of the expandable framework 110 within the native valve annulus. Positioning the replacement heart valve implant 100 at the desired insertion depth 102 and/or within a maximum tolerance from the desired insertion depth 102, the replacement heart valve implant 100 and/or the expandable framework 110 may exhibit optimal arching within the native valve annulus and thereby prevent migration of the replacement heart valve implant 100 and/or the expandable framework 110 downstream (or upstream).

Positioning the replacement heart valve implant 100 and/or the expandable framework 110 within the native valve annulus may be accomplished by locating the primary visual indicator 68 within the native valve annulus and/or aligned with the reference plane A associated with the native valve annulus, as shown in FIG. 3. During visualization, the aortic valve 12 may be identified and/or visualized under fluoroscopy using known means and/or methods, such as contrast injection. Once the aortic valve 12 has been identified, the reference plane A associated with the native valve annulus may also be identified.

In some embodiments, the primary visual indicator 68 may be configured to define an actual insertion depth of the replacement heart valve implant 100 relative to the reference plane A associated with the native valve annulus. The primary visual indicator 68 may be very effective for properly locating the replacement heart valve implant 100 relative to the reference plane A associated with the native valve annulus, when the view from the imaging device 300 is aligned with the reference plane A associated with the native valve annulus.

Unfortunately, it is possible for the imaging device 300 to be misaligned with and/or with respect to the reference plane A associated with the native valve annulus, as shown in FIG. 4. If this occurs, the accuracy of the placement of the replacement heart valve implant 100 may be compromised. It may be possible for the actual insertion depth to be off and/or incorrect by up to 5 millimeters if the imaging device 300 is misaligned with and/or with respect to the reference plane A associated with the native valve annulus.

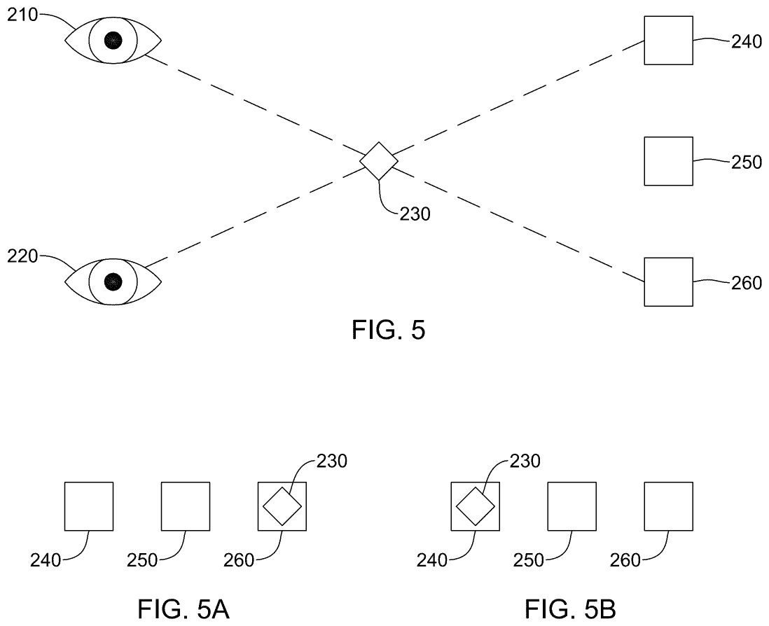

Misalignment of the imaging device 300 with and/or with respect to the reference plane A associated with the native valve annulus may induce the parallax effect during visualization. The parallax effect is described schematically with respect to FIG. 5. For reference, if the imaging device 300 is aligned with the reference plane A associated with the native valve annulus, a target 230 (e.g., the primary visual indicator 68) may appear aligned with a correct position 250 (e.g., the native valve annulus and/or the reference plane A associated with the native valve annulus). If the imaging device 300 is positioned at an oblique angle to and/or relative to the reference plane A associated with the native valve annulus, the parallax effect may be observed. The greater the misalignment of the imaging device 300 with the reference plane A associated with the native valve annulus, the further the actual insertion depth may deviate from the desired insertion depth 102.

In one example of the parallax effect, if the imaging device 300 is positioned proximally of the reference plane A associated with the native valve annulus, the imaging device 300 may provide a proximal viewpoint 210 that shifts the apparent position of the target 230 (e.g., the primary visual indicator 68) to a distal position 260 relative to the correct position 250 (e.g., the native valve annulus and/or the reference plane A associated with the native valve annulus), as seen in FIG. 5A. As a result of this view, the user may understand the primary visual indicator 68 to be positioned too deeply into the left ventricle and may retract the implant delivery system 30 until the primary visual indicator 68 appears to be aligned with the reference plane A associated with the native valve annulus before deploying the replacement heart valve implant 100. Effectively, while the primary visual indicator 68 may be correctly located in alignment with the reference plane A associated with the native valve annulus, the parallax effect will make it appear as though the primary visual indicator 68 is not in correct alignment with the reference plane A associated with the native valve annulus. As such, in reality, the parallax effect associated with the proximal viewpoint 210 will cause the replacement heart valve implant 100 to be positioned too far downstream relative to the native valve annulus.

In another example of the parallax effect, if the imaging device 300 is positioned distally of the reference plane A associated with the native valve annulus, the imaging device 300 may provide a distal viewpoint 220 that shifts the apparent position of the target 230 (e.g., the primary visual indicator 68) to a proximal position 240 relative to the correct position 250 (e.g., the native valve annulus and/or the reference plane A associated with the native valve annulus), as seen in FIG. 5B. As a result of this view, the user may understand the primary visual indicator 68 to be positioned not deep enough into the left ventricle and may advance the implant delivery system 30 until the primary visual indicator 68 appears to be aligned with the reference plane A associated with the native valve annulus before deploying the replacement heart valve implant 100. Effectively, while the primary visual indicator 68 may be correctly located in alignment with the reference plane A associated with the native valve annulus, the parallax effect will make it appear as though the primary visual indicator 68 is not in correct alignment with the reference plane A associated with the native valve annulus. As such, in reality, the parallax effect associated with the distal viewpoint 220 will cause the replacement heart valve implant 100 to be positioned too far upstream relative to the native valve annulus.

When the imaging device 300 is properly aligned with the reference plane A associated with the native valve annulus, the imaging device 300 may provide a viewpoint disposed between the proximal viewpoint 210 and the distal viewpoint 220 that aligns the target 230 (e.g., the primary visual indicator 68) with the correct position 250 (e.g., the native valve annulus and/or the reference plane A associated with the native valve annulus). When the imaging device 300 is correctly aligned with the reference plane A associated with the native valve annulus, the actual insertion depth will align with the desired insertion depth 102 and the replacement heart valve implant 100 will be correctly positioned within the native valve annulus.

In view of the difficulties that may be created by the parallax effect, procedure effectiveness and/or patient safety may be compromised without additional costly and/or time-consuming visualization verification steps. This disclosure relates to apparatus and methods developed to reduce the need for those additional costly and/or time-consuming visualization verification steps by reducing and/or eliminating the possibility of the parallax effect during delivery of the replacement heart valve implant 100 to the native valve annulus.

FIG. 6 is a partial cross-sectional view illustrating selected aspects of the implant delivery system 30 of FIG. 2 in accordance with the disclosure. For improved clarity, the replacement heart valve implant 100 and some portions of the implant delivery system 30 are not shown. For example, in FIG. 6, the distal sheath 54 is shown while the proximal sheath 52 is not shown.

As seen in FIG. 6, the elongate shaft assembly 50 may include the primary visual indicator 68. In some embodiments, the primary visual indicator 68 may be fixedly attached to the elongate shaft assembly 50. In some embodiments, the primary visual indicator 68 may be a marker band embedded within the elongate shaft assembly 50. Other configurations are also contemplated. As discussed herein, the primary visual indicator 68 may be configured and/or adapted to be visible under fluoroscopy with the imaging device 300.

In some embodiments, the implant delivery system 30 and/or the elongate shaft assembly 50 may include the secondary visual indicator 78. In some embodiments, the secondary visual indicator 78 may be fixedly attached to the elongate shaft assembly 50. In some embodiments, the primary visual indicator 68 may be axially spaced apart from the secondary visual indicator 78. The secondary visual indicator 78 may be configured and/or adapted to be visible under fluoroscopy with the imaging device 300 when the imaging device 300 is aligned with the reference plane A associated with the native valve annulus. The secondary visual indicator 78 may be configured and/or adapted to be not visible under fluoroscopy with the imaging device 300 when the imaging device 300 is misaligned with the reference plane A associated with the native valve annulus.

In some embodiments, the implant delivery system 30 and/or the elongate shaft assembly 50 may include the stent holder 70 configured to engage the expandable framework 110 of the replacement heart valve implant 100 in the radially collapsed configuration and/or when the replacement heart valve implant 100 is constrained within the implant holding portion of the implant delivery system 30. The stent holder 70 is shown in more detail in FIGS. 7A and 7B. The stent holder 70 may include a first end portion 72, a central cylindrical portion 74, and a second end portion 76 disposed opposite the first end portion 72. In some embodiments, the first end portion 72 may have a generally bulbous shape. In some embodiments, the stent holder 70 may be configured and/or adapted to be visible under fluoroscopy. In some embodiments, the stent holder 70 may be formed from stainless steel. Some suitable but non-limiting materials for the stent holder 70 and/or components or elements thereof, for example metallic materials and/or polymeric materials, are described below.

In some embodiments, an outermost radial extent of the first end portion 72 of the stent holder 70 may be disposed proximate a distal end of the first end portion 72 of the stent holder 70. In some embodiments, the first end portion 72 of the stent holder 70 may be tapered radially inward in a proximal direction from the outermost radial extent of the stent holder 70. In some embodiments, the stent holder 70 may include a lumen extending longitudinally and/or axially therethrough. In at least some embodiments, at least a portion of the elongate shaft assembly 50 may extend longitudinally and/or axially through the lumen of the stent holder 70.

The first end portion 72 may be configured and/or adapted to engage the expandable framework 110 of the replacement heart valve implant 100 in the radially collapsed configuration and/or when the replacement heart valve implant 100 is constrained within the implant holding portion of the implant delivery system 30. In some embodiments, the first end portion 72 may include at least one projection 73 configured and/or adapted to engage the expandable framework 110 of the replacement heart valve implant 100 in the radially collapsed configuration and/or when the replacement heart valve implant 100 is constrained within the implant holding portion of the implant delivery system 30. In some embodiments, the at least one projection 73 may extent radially outward from the first end portion 72 of the stent holder 70. In some embodiments, the at least one projection 73 may extend radially outward through the expandable framework 110 of the replacement heart valve implant 100 in the radially collapsed configuration and/or when the replacement heart valve implant 100 is constrained within the implant holding portion of the implant delivery system 30.

In some embodiments, the second end portion 76 may include a plurality of fingers 77 extending longitudinally and/or parallel to a central longitudinal axis of the elongate shaft assembly 50. In some embodiments, the plurality of fingers 77 may extend away from the first end portion 72 and/or the central cylindrical portion 74. In some embodiments, the plurality of fingers 77 may be longitudinally and/or axially aligned with the at least one projection 73. In some embodiments, the plurality of fingers 77 may be configured and/or adapted to align with the plurality of commissures of the replacement heart valve implant 100. In some embodiments, the plurality of fingers 77 may be configured and/or adapted to be visible under fluoroscopy. In some embodiments, the plurality of fingers 77 may be configured and/or adapted to align the implant delivery system 30, the stent holder 70, and/or the replacement heart valve implant 100 with native valve commissures of the aortic valve 12 under fluoroscopy. In some embodiments, the plurality of fingers 77 may be configured and/or adapted to rotationally align the implant delivery system 30, the stent holder 70, and/or the replacement heart valve implant 100 with native valve commissures of the aortic valve 12 under fluoroscopy.

In some embodiments, the secondary visual indicator 78 may comprise an annular recess formed within the stent holder 70, as seen in FIGS. 7A and 7B. In some embodiments, the secondary visual indicator 78 and/or the annular recess may be formed within the central cylindrical portion 74 of the stent holder 70. In some embodiments, the secondary visual indicator 78 and/or the annular recess may be configured and/or adapted to be visible to the imaging device 300 as a void under fluoroscopy. In some embodiments, the secondary visual indicator 78 and/or the annular recess may have a width W of about 0.3 millimeters to about 0.4 millimeters. In some embodiments, the secondary visual indicator 78 and/or the annular recess may have a radial depth D defining a remaining radial material thickness T of 0.07+/−0.01 millimeters. As such, after forming the secondary visual indicator 78 and/or the annular recess, the remaining radial material thickness T between the lumen of the stent holder 70 and a radially outward facing surface of the annular recess is 0.07+/−0.01 millimeters. Other configurations and/or dimensions are also contemplated. In some embodiments, the remaining radial material thickness T may be sized, configured, and/or adapted to perform mechanically while being thin enough that the secondary visual indicator 78 appears as a void under fluoroscopy when the imaging device 300 is properly aligned with the reference plane A associated with the native valve annulus.

Returning to FIG. 6, in some embodiments, the implant delivery system 30 and/or the elongate shaft assembly 50 may include the distal tip 58 and the distal sheath 54. The distal sheath 54 may extend proximally from the distal tip 58. The implant delivery system may include a distal cap 60 disposed distal of the stent holder 70 and/or disposed between the stent holder 70 and the distal tip 58. The implant delivery system and/or the implant holding portion may include a cage 62 extending radially outward from the elongate shaft assembly 50, the distal cap 60 and/or the stent holder 70. In some embodiments, the distal cap 60 may be configured to axially retain the cage 62 over the second end portion 76 of the stent holder 70. In some embodiments, the cage 62 may be configured to substantially center the distal sheath 54 over and/or around the elongate shaft assembly 50 as the distal sheath 54 is moved from an open configuration to the closed configuration. Other configurations are also contemplated.

In some embodiments, the implant delivery system 30 and/or the implant holding portion may include an atraumatic transition shield 80. The atraumatic transition shield 80 may be disposed adjacent the stent holder 70. In some embodiments, the atraumatic transition shield 80 may be disposed between the stent holder 70 and the handle 40. In some embodiments, the atraumatic transition shield 80 may be disposed proximal the stent holder 70. In some embodiments, the atraumatic transition shield 80 may be disposed at and/or adjacent the first end portion 72 of the stent holder 70. In some embodiments, the atraumatic transition shield 80 may axially overlap the first end portion 72 of the stent holder 70. In some embodiments, the atraumatic transition shield 80 may be disposed radially outward of at least a portion of the first end portion 72 of the stent holder 70. In some embodiments, the atraumatic transition shield 80 may be tapered radially inward in the proximal direction and/or toward the handle 40. The atraumatic transition shield 80 may be configured to prevent the replacement heart valve implant 100, the expandable framework 110, the plurality of valve leaflets 120, etc. from catching on the stent holder 70 as the implant delivery system 30 is withdrawn after deploying the replacement heart valve implant 100.

In use, after navigating the implant delivery system 30 and/or the implant holding portion to the treatment site (over a guidewire 90 (e.g., FIG. 9) for example), the proximal sheath 52 and/or the distal sheath 54 may be axially translated relative to each other to shift the implant holding portion to the open configuration. When unconstrained by the implant holding portion, the replacement heart valve implant 100 and/or the expandable framework 110 may be configured to shift from the radially collapsed configuration to the radially expanded configuration. Shifting the replacement heart valve implant 100 and/or the expandable framework 110 toward the radially expanded configuration after axially translating the proximal sheath 52 and/or the distal sheath 54 away from each other and/or the stent holder 70 may permit the replacement heart valve implant 100 and/or the expandable framework 110 to decouple and/or detach from the implant delivery system 30. Some suitable but non-limiting materials for the implant delivery system 30, the handle 40, the elongate shaft assembly 50, the proximal sheath 52, the distal sheath 54, the primary visual indicator 68, the atraumatic transition shield 80, and/or components or elements thereof, for example metallic materials and/or polymeric materials, are described below.

In at least some interventions, the replacement heart valve implant 100 may be deployed within the native heart valve (e.g., the native heart valve is left in place and not excised). Alternatively, the native heart valve may be removed (such as through valvuloplasty, for example) and the replacement heart valve implant 100 may be deployed in its place as a replacement.

FIGS. 8A, 8B, and 9 are fluoroscopic images illustrating selected aspects of the implant delivery system 30. For reference, FIG. 8A corresponds to an image as viewed from the imaging device 300 when the imaging device 300 is aligned with the reference plane A associated with the native valve annulus (e.g., as in FIG. 3), and FIG. 8B corresponds to an image as viewed from the imaging device 300 when the imaging device 300 is misaligned with the reference plane A associated with the native valve annulus (as in FIG. 4). It should be noted that the images shown in FIGS. 8A and 8B were taken using a reference structure to improve clarity, and thus actual patient anatomy is not visible in these images. Similarly, the replacement heart valve implant 100 is also not shown in FIGS. 8A and 8B. The distal sheath 54, the distal tip 58, and the stent holder 70 are identified in the figures as reference points in FIGS. 8A and 8B. FIG. 9 includes additional elements of the patient's anatomy and the replacement heart valve system to further illustrate selected aspects of the disclosure.

As seen in FIGS. 8A and 9, the secondary visual indicator 78 may be configured and/or adapted to be visible under fluoroscopy with the imaging device 300 when the imaging device is aligned with the reference plane A associated with the native valve annulus. In some embodiments, when the imaging device 300 is aligned with the reference plane A associated with the native valve annulus and the primary visual indicator 68 is aligned with the reference plane A associated with the native valve annulus under fluoroscopy, the secondary visual indicator 78 and/or the annular recess is visible to the imaging device 300 as a void oriented generally parallel to the reference plane A associated with the native valve annulus.

In some embodiments, when the secondary visual indicator 78 is visible under fluoroscopy and the primary visual indicator 68 is aligned with the reference plane A associated with the native valve annulus under fluoroscopy, the actual insertion depth may be within 10% of the desired insertion depth 102 (e.g., FIG. 1). In some embodiments, when the secondary visual indicator 78 is visible under fluoroscopy and the primary visual indicator 68 is aligned with the reference plane A associated with the native valve annulus under fluoroscopy, the actual insertion depth may be within 7.5% of the desired insertion depth 102. In some embodiments, when the secondary visual indicator 78 is visible under fluoroscopy and the primary visual indicator 68 is aligned with the reference plane A associated with the native valve annulus under fluoroscopy, the actual insertion depth may be within 5% of the desired insertion depth 102. In some embodiments, when the secondary visual indicator 78 is visible under fluoroscopy and the primary visual indicator 68 is aligned with the reference plane A associated with the native valve annulus under fluoroscopy, the actual insertion depth may be within 2.5% of the desired insertion depth 102. In some embodiments, when the secondary visual indicator 78 is visible under fluoroscopy and the primary visual indicator 68 is aligned with the reference plane A associated with the native valve annulus under fluoroscopy, the actual insertion depth may be within 1% of the desired insertion depth 102. Other configurations are also contemplated.

As seen in FIG. 8B, the secondary visual indicator 78 is not visible under fluoroscopy with the imaging device 300 when the imaging device is misaligned with the reference plane A associated with the native valve annulus. In some embodiments, when the imaging device 300 is misaligned with the reference plane A associated with the native valve annulus and the primary visual indicator 68 is aligned with the reference plane A associated with the native valve annulus under fluoroscopy, the secondary visual indicator 78 and/or the annular recess is at least partially obscured from viewing the imaging device 300.

In some embodiments, when the secondary visual indicator 78 is not visible under fluoroscopy and the primary visual indicator 68 is aligned with the reference plane A associated with the native valve annulus under fluoroscopy, the actual insertion depth may deviate from the desired insertion depth 102 (e.g., FIG. 1) by at least 1%. In some embodiments, when the secondary visual indicator 78 is not visible under fluoroscopy and the primary visual indicator 68 is aligned with the reference plane A associated with the native valve annulus under fluoroscopy, the actual insertion depth may deviate from the desired insertion depth 102 (e.g., FIG. 1) by at least 2.5%. In some embodiments, when the secondary visual indicator 78 is not visible under fluoroscopy and the primary visual indicator 68 is aligned with the reference plane A associated with the native valve annulus under fluoroscopy, the actual insertion depth may deviate from the desired insertion depth 102 (e.g., FIG. 1) by at least 5%. In some embodiments, when the secondary visual indicator 78 is not visible under fluoroscopy and the primary visual indicator 68 is aligned with the reference plane A associated with the native valve annulus under fluoroscopy, the actual insertion depth may deviate from the desired insertion depth 102 (e.g., FIG. 1) by at least 7.5%. In some embodiments, when the secondary visual indicator 78 is not visible under fluoroscopy and the primary visual indicator 68 is aligned with the reference plane A associated with the native valve annulus under fluoroscopy, the actual insertion depth may deviate from the desired insertion depth 102 (e.g., FIG. 1) by at least 10%.

In some embodiments, when the primary visual indicator 68 is aligned with the reference plane A associated with the native valve annulus, the secondary visual indicator 78 is only visible to the imaging device 300 under fluoroscopy when the imaging device 300 is aligned with the reference plane A associated with the native valve annulus. In some embodiments, when the primary visual indicator 68 is aligned with the reference plane A associated with the native valve annulus, the secondary visual indicator 78 is only visible to the imaging device 300 under fluoroscopy when the imaging device 300 is not oriented at an oblique angle intersecting the reference plane A associated with the native valve annulus. In some cases, a variation and/or deviation of the imaging device 300 of as little as 5 degrees from the reference plane A associated with the native valve annulus may cause the secondary visual indicator 78 to disappear from view under fluoroscopy, the secondary visual indicator 78 being obscured by the rest of the stent holder 70 (e.g., the first end portion 72 and/or the central cylindrical portion 74).

When the primary visual indicator 68 is aligned with the reference plane A associated with the native valve annulus, and the secondary visual indicator 78 is visible to the imaging device 300, the primary visual indicator 68 may be accurately and/or reliably used to position the replacement heart valve implant 100 such that the actual insertion depth is within an acceptable tolerance of the desired insertion depth 102. In some embodiments, the acceptable tolerance may be about 10%, about 7.5%, about 5%, about 2.5%, about 1%, or another suitable amount determined by testing and/or trial.

In some embodiments, the implant delivery system 30 is configured to position the replacement heart valve implant 100 within the native valve annulus at an actual insertion depth relative to the reference plane A associated with the native valve annulus that is within 10% of the desired insertion depth relative to the reference plane A associated with the native valve annulus when the primary visual indicator 68 and the secondary visual indicator 78 are both visible to the imaging device 300 under fluoroscopy. In some embodiments, the implant delivery system 30 is configured to position the replacement heart valve implant 100 within the native valve annulus at an actual insertion depth relative to the reference plane A associated with the native valve annulus that is within 7.5% of the desired insertion depth relative to the reference plane A associated with the native valve annulus when the primary visual indicator 68 and the secondary visual indicator 78 are both visible to the imaging device 300 under fluoroscopy. In some embodiments, the implant delivery system 30 is configured to position the replacement heart valve implant 100 within the native valve annulus at an actual insertion depth relative to the reference plane A associated with the native valve annulus that is within 5% of the desired insertion depth relative to the reference plane A associated with the native valve annulus when the primary visual indicator 68 and the secondary visual indicator 78 are both visible to the imaging device 300 under fluoroscopy. In some embodiments, the implant delivery system 30 is configured to position the replacement heart valve implant 100 within the native valve annulus at an actual insertion depth relative to the reference plane A associated with the native valve annulus that is within 2.5% of the desired insertion depth relative to the reference plane A associated with the native valve annulus when the primary visual indicator 68 and the secondary visual indicator 78 are both visible to the imaging device 300 under fluoroscopy. In some embodiments, the implant delivery system 30 is configured to position the replacement heart valve implant 100 within the native valve annulus at an actual insertion depth relative to the reference plane A associated with the native valve annulus that is within 1% of the desired insertion depth relative to the reference plane A associated with the native valve annulus when the primary visual indicator 68 and the secondary visual indicator 78 are both visible to the imaging device 300 under fluoroscopy.

In some embodiments, a method of delivering the replacement heart valve implant 100 to the native valve annulus may comprise advancing the implant delivery system 30 to a position adjacent the native valve annulus. In some embodiments, the method of delivering the replacement heart valve implant 100 to the native valve annulus may comprise positioning the imaging device 300 in alignment with the reference plane A associated with the native valve annulus. In some embodiments, the method of delivering the replacement heart valve implant 100 to the native valve annulus may comprise aligning the primary visual indicator 68 of the implant delivery system 30 with the reference plane A associated with the native valve annulus under fluoroscopy using the imaging device 300. In some embodiments, the method of delivering the replacement heart valve implant 100 to the native valve annulus may comprise visualizing the secondary visual indicator 78 of the implant delivery system 30 under fluoroscopy using the imaging device 300 when the primary visual indicator 68 is aligned with the reference plane A associated with the native valve annulus to verify alignment of the imaging device 300 with the reference plane A associated with the native valve annulus. In some embodiments, the method of delivering the replacement heart valve implant 100 to the native valve annulus may comprise deploying the replacement heart valve implant 100 from the implant delivery system 30 within the native valve annulus after verifying alignment of the imaging device 300 with the reference plane A associated with the native valve annulus and after aligning the primary visual indicator 68 with the reference plane A associated with the native valve annulus.

The materials that can be used for the various components of the replacement heart valve system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the replacement heart valve implant, the expandable framework, the plurality of valve leaflets, the implant delivery system, the handle, the elongate shaft assembly, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluorocthylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-clastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, cPTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An implant delivery system for delivering a replacement heart valve implant to a native valve annulus, comprising:
an elongate shaft assembly including an implant holding portion comprising a proximal sheath and a distal sheath;
wherein the implant holding portion is configured to constrain a replacement heart valve implant in a radially collapsed configuration;
wherein the elongate shaft assembly includes a primary visual indicator configured to be visible under fluoroscopy with an imaging device;
wherein the elongate shaft assembly includes a secondary visual indicator configured to be visible under fluoroscopy with the imaging device when the imaging device is aligned with a reference plane associated with the native valve annulus;
wherein the secondary visual indicator is not visible under fluoroscopy with the imaging device when the imaging device is misaligned with the reference plane associated with the native valve annulus.

2. The implant delivery system of claim 1, wherein a desired insertion depth of the replacement heart valve implant relative to the reference plane associated with the native valve annulus is about 7 millimeters from the reference plane associated with the native valve annulus to an inflow end of the replacement heart valve implant.

3. The implant delivery system of claim 2, wherein the primary visual indicator is configured to define an actual insertion depth of the replacement heart valve implant relative to the reference plane associated with the native valve annulus.

4. The implant delivery system of claim 3, wherein when the secondary visual indicator is visible under fluoroscopy and the primary visual indicator is aligned with the reference plane under fluoroscopy, the actual insertion depth is within 10% of the desired insertion depth.

5. The implant delivery system of claim 3, wherein when the secondary visual indicator is not visible under fluoroscopy and the primary visual indicator is aligned with the reference plane under fluoroscopy, the actual insertion depth deviates from desired insertion depth by at least 10%.

6. The implant delivery system of claim 1, wherein the elongate shaft assembly includes a stent holder configured to engage an expandable framework of the replacement heart valve implant in the radially collapsed configuration.

7. The implant delivery system of claim 6, wherein the secondary visual indicator comprises an annular recess formed within the stent holder.

8. The implant delivery system of claim 7, wherein when the imaging device is aligned with the reference plane associated with the native valve annulus and the primary visual indicator is aligned with the reference plane under fluoroscopy, the annular recess is visible to the imaging device as a void oriented generally parallel to the reference plane.

9. The implant delivery system of claim 7, wherein when the imaging device is misaligned with the reference plane associated with the native valve annulus and the primary visual indicator is aligned with the reference plane under fluoroscopy, the annular recess is at least partially obscured from viewing by the imaging device.

10. The implant delivery system of claim 7, wherein the annular recess has a width of 0.3 millimeters to 0.4 millimeters.

11. The implant delivery system of claim 10, wherein the annular recess has a radial depth defining a remaining radial material thickness of 0.07+/−0.01 millimeters.

12. The implant delivery system of claim 1, wherein the primary visual indicator is axially spaced apart from the secondary visual indicator.

13. A replacement heart valve system, comprising:
a replacement heart valve implant comprising an expandable framework and a plurality of valve leaflets secured to the expandable framework, the expandable framework being configured to shift between a radially collapsed configuration and a radially expanded configuration; and
an implant delivery system for delivering the replacement heart valve implant to a native valve annulus, the implant delivery system comprising:
an elongate shaft assembly including an implant holding portion comprising a proximal sheath and a distal sheath;
wherein the implant holding portion is configured to constrain the replacement heart valve implant in the radially collapsed configuration;
wherein the elongate shaft assembly includes a primary visual indicator configured to be visible under fluoroscopy with an imaging device;
wherein the elongate shaft assembly includes a secondary visual indicator configured to be visible under fluoroscopy with the imaging device when the imaging device is aligned with a reference plane associated with the native valve annulus;
wherein the secondary visual indicator is not visible under fluoroscopy with the imaging device when the imaging device is misaligned with the reference plane associated with the native valve annulus.

14. The replacement heart valve system of claim 13, wherein the implant delivery system is configured to cooperate with the imaging device to position the replacement heart valve implant within the native valve annulus.

15. The replacement heart valve system of claim 13, wherein the implant delivery system is configured to position the replacement heart valve implant within the native valve annulus at an actual insertion depth relative to the reference plane associated with native valve annulus that is within 10% of a desired insertion depth relative to the reference plane associated with native valve annulus when the primary visual indicator and the secondary visual indicator are both visible to the imaging device under fluoroscopy.

16. The replacement heart valve system of claim 15, wherein when the primary visual indicator is aligned with the reference plane, the secondary visual indicator is only visible to the imaging device under fluoroscopy when the imaging device is aligned with the reference plane associated with the native valve annulus.

17. A method of delivering a replacement heart valve implant to a native valve annulus, comprising:
advancing an implant delivery system to a position adjacent the native valve annulus, wherein the replacement heart valve implant is constrained within an implant holding portion of the implant delivery system;
positioning an imaging device in alignment with a reference plane associated with the native valve annulus;
aligning a primary visual indicator of the implant delivery system with the reference plane under fluoroscopy using the imaging device;
visualizing a secondary visual indicator of the implant delivery system under fluoroscopy using the imaging device when the primary visual indicator is aligned with the reference plane to verify alignment of the imaging device with the reference plane; and
deploying the replacement heart valve implant from the implant delivery system within the native valve annulus.

18. The method of claim 17, wherein the secondary visual indicator is configured to be visible under fluoroscopy with the imaging device when the imaging device is aligned with the reference plane associated with the native valve annulus and the primary visual indicator is aligned with the reference plane, and the secondary visual indicator is not visible under fluoroscopy with the imaging device when the imaging device is misaligned with the reference plane associated with the native valve annulus and the primary visual indicator is aligned with the reference plane.

19. The method of claim 18, wherein when the primary visual indicator is aligned with the reference plane, the secondary visual indicator is only visible under fluoroscopy with the imaging device when the imaging device is not oriented at an oblique angle intersecting the reference plane associated with the native valve annulus.

20. The method of claim 17, wherein the secondary visual indicator is an annular recess formed in a stent holder of the implant delivery system, the stent holder being configured to engage an expandable framework of the replacement heart valve implant when the replacement heart valve implant is constrained within the implant holding portion.
